(19) United States
(12) Patent Application Publication
(10) Pub. No.: US 2007/0042452 A1
(43) Pub. Date: Feb. 22, 2007

(54) Detection of plasma ACE2

(76) Inventors: Rebecca Ann Lew, Port Melbourne (AU); Alexander Ian Smith, Hampton (AU); Louise Mary Burrell, Brunswick (AU)

Correspondence Address: SHERIDAN ROSS PC, 1560 BROADWAY, SUITE 1200, DENVER, CO, 80202, US

(21) Appl. No.: 11/398,499

(22) Filed: Apr. 4, 2006

Related U.S. Application Data
(60) Provisional application No. 60/668,310, filed on Apr. 4, 2005.

(51) Int. Cl. C12Q 1/37

(52) U.S. Cl. 435/23

(57) ABSTRACT

A method of assessing a subject for aberrant cardiac function is described. The method particularly involves the measurement of a plasma biomarker, namely metallopeptidase angiotensin-converting enzyme 2 (ACE2), implicated in ischaemic heart disease.

### CROSS-REFERENCE TO RELATED APPLICATIONS

This application claims priority under 35 U.S.C. § 119(e) to U.S. Provisional Patent Application Ser. No. 60/668,310 filed Apr. 4, 2005, which is incorporated herein in its entirety by this reference.

### FIELD OF THE INVENTION

The present invention relates to a method of assessing a subject for aberrant cardiac function. In particular, the method involves the measurement of a plasma biomarker implicated in ischaemic heart disease.

### BACKGROUND OF THE INVENTION

The generation of the vasoconstrictor peptide angiotensin II (Ang II) by the renin-angiotensin system (RAS) is recognised as a critical point in the regulation of cardiovascular function. The final step in the production of Ang II is catalysed by the metallopeptidase angiotensin-converting enzyme (ACE), and inhibitors of ACE have been well-established as the basis of therapies for hypertension, heart failure, and myocardial infarction (MI).

Five years ago, a homologous enzyme known as ACE2 (displaying approximately 40% sequence homology to ACE) was discovered, and shortly thereafter, an ACE2 knock-out mouse was developed. This animal was found to display severe defects in cardiac contractility, thereby indicating a crucial role for ACE2 in the function of the heart.

Unlike ACE, ACE2 acts strictly as a carboxypeptidase, removing a single C-terminal residue from a limited range of peptides. Indeed, both in vitro and in vivo evidence suggests that Ang II is the most likely substrate for ACE2; removal of the C-terminal phenylalanyl residue not only inactivates Ang II, but also produces the putative vasodilatory peptide, Ang 1-7.

Thus, the catalytic characteristics of ACE2 suggest that it may serve in a counter-regulatory manner to ACE, by inactivating Ang II and generating Ang 1-7.

ACE2 expression is essentially confined to endothelial cells of the coronary and renal vasculatures, the renal tubule epithelium, and Leydig cells of the testis. In both rat and human cardiac tissue following MI, expression of ACE2 has been found to be markedly upregulated, which may indicate either a protective or regenerative role for the enzyme and its substrate(s). Preliminary evidence suggests that ACE2 is released into plasma following MI, and thus may be useful as a potential biomarker for aberrant cardiac function such as myocardial ischaemia and infarction. However, while ACE has been found to be proteolytically released from its usual endothelial cell surface location (Ramchandran et al, 1996) into biological fluids such as plasma, urine, cerebrospinal fluid and semen (in a process known as “shedding”) and can therefore be used as a plasma biomarker for certain diseases (eg sarcoidosis), there have been no previous reports of the detection of ACE2 in such biological fluids or the use of ACE2 as a biomarker of human disease.

In work leading to the present invention, the present applicants have found that there exists in plasma, an inhibitor or “masking agent” of ACE2 which has hithertobefore prevented the detection of ACE2 in plasma. Using chromatography to extract the ACE2 from plasma, thereby allowing the measurement of plasma ACE2 levels, the present applicants have subsequently been able to show that there is a marked elevation in the plasma ACE2 levels of humans having suffered MI. Accordingly, the measurement of plasma ACE2 levels shows considerable promise as the basis of a diagnostic method for assessing a subject for aberrant cardiac function or as a prognostic method for predicting outcome following myocardial infarction.

### SUMMARY OF THE INVENTION

The present invention therefore provides a method of assessing a subject for aberrant cardiac function, said method comprising the steps of:
(i) providing a sample from said subject;
(ii) treating said sample to
   separate any metallopeptidase angiotensin-converting enzyme 2 (ACE2) present in said sample from ACE2 inhibiting agent(s), or otherwise to
   remove or inactivate ACE2 inhibiting agent(s); and
(iii) detecting the level of ACE2 in said treated sample,
   wherein detection of an elevated level of ACE2 in said treated sample, relative to typical levels of ACE2 found in identical samples from healthy subjects, is indicative of aberrant cardiac function.

In a second aspect of the present invention, there is provided a process of separating metallopeptidase angiotensin-converting enzyme 2 (ACE2) from ACE2 inhibiting agent(s) in a sample from a subject, said process comprising the use of chromatography to separate any ACE2 present in said sample from ACE2 inhibiting agent(s).

In a third aspect, the present invention provides a kit for detecting the level of metallopeptidase angiotensin-converting enzyme 2 (ACE2) in a sample from a subject, said kit comprising:
(i) means for separating any ACE2 present in said sample from any ACE2 inhibiting agent(s) in said sample, or otherwise for removing or inactivating any ACE2 inhibiting agent(s) in said sample; and
(ii) means for detecting the level of ACE2 in said sample.

In a fourth aspect, the present invention provides a method of assessing a subject for aberrant cardiac function by use of a kit according to the third aspect of the invention.

### BRIEF DESCRIPTION OF THE FIGURES

FIG. 1 provides graphical results showing that the catalytic activity of recombinant soluble ACE2 expressed from CHO cells was inhibited by the addition of plasma. The extent of inhibition was both dose-and time-dependent, indicating the presence in the plasma of a competitive inhibiting agent.

FIG. 2 provides a bar graph of the catalytic activity of ACE2 detected in the medium of CHO cells expressing full-length human ACE2. ACE2 release from the plasma membrane of CHO cells was increased by the addition of phorbol ester (PMA 1 μM). N=3 per group.

FIG. 3A provides an immunological localisation of ACE2 in the heart following cardiac injury, and FIG. 3B shows preliminary data indicating the increase in plasma ACE2 activity after myocardial infarction. Individual values are shown as circles; the average for each group is indicated by a line. Note that activity (expressed as ng ACE2/mL plasma) is plotted on a logarithmic scale.

FIG. 4 provides a bar graph showing that plasma ACE2 levels are elevated in subjects who have experienced acute myocardial infarction (AMI) as compared to subjects who have entered the clinic to undergo percutaneous coronary intervention (PCI, “angioplasty”) with no indication of myocardial infarction. Data are expressed as ng ACE2/mL plasma; p<0.05 as determined by analysis of variance.

### DETAILED DESCRIPTION OF THE INVENTION

The present applicants have identified a method for chromatographically extracting ACE2 from a plasma sample of a subject to allow for the measurement of ACE2 levels in plasma. The chromatographic method results in the separation of any ACE2 present in the plasma sample from an inhibiting agent(s) of ACE2 which has hithertobefore prevented the detection of ACE2 in plasma. The present applicants have also found that plasma ACE2 levels are markedly elevated after myocardial infarction (MI), up to 20-fold greater than normal. This elevation in plasma ACE2 levels indicates a specific response to ischaemia or other cardiac injury.

In a first aspect of the present invention, there is provided a method of assessing a subject for aberrant cardiac function, said method comprising the steps of:
(i) providing a sample from said subject;
(ii) treating said sample to
   separate any metallopeptidase angiotensin-converting enzyme 2 (ACE2) present in said sample from ACE2 inhibiting agent(s), or otherwise to
   remove or inactivate ACE2 inhibiting agent(s); and
(iii) detecting the level of ACE2 in said treated sample,
   wherein detection of an elevated level of ACE2 in said treated sample, relative to typical levels of ACE2 found in identical samples from healthy subjects, is indicative of aberrant cardiac function.

Preferably, the sample from said subject is a plasma sample, however it is considered that the method of the invention may be equally applicable to other biological samples, particularly whole blood, lymph, urine, cerebrospinal fluid, and semen.

As used herein, the term “plasma” refers to the fluid fraction of blood, and includes, for example, blood that has been treated with a coagulant and centrifuged to remove the cells (ie serum).

As used herein, the term “ACE2 inhibiting agent” refers to an endogenous agent present in, at least, blood and plasma, which inhibits or prevents detection of ACE2 activity, particularly through measurement of cleavage or consumption of a detectably-labelled substrate of ACE2 (eg the quenched fluorescent substrate (QFS), (7-methoxycoumarin-4-yl)-acetyl-Ala-Pro-Lys(2,4-dinitrophenyl)).

Preferably, the step of treating the sample to either separate any ACE2 present in the sample from ACE2 inhibiting agent(s), or otherwise to remove or inactivate ACE2 inhibiting agent(s), involves the use of chromatography to separate any ACE2 present in the sample from ACE2 inhibiting agent(s).

Suitable methods of chromatography which may be used in the method of the invention such as chromatographic methods for separating components of a mixture by differential adsorption of compounds to adsorbents, partitioning between stationary and mobile immiscible phases, ion exchange, or a combination of any of these, will be well known to persons skilled in the art. Particular examples of such chromatographic methods include adsorption, affinity, affinity-elution, ampholyte displacement, ascending biospecific-elution, charge-transfer, circular, counter-current, covalent, descending, dye-ligand, electro, exclusion, frontal, gas-liquid, gel-filtration, gel-permeation, high performance liquid chromatography (HPLC), liquid-affinity, high-pressure liquid, hydrophobic, ion-exchange, ion-exclusion, ionic-interaction, molecular exclusion, molecular sieve, partition, permeation, positive and immunochromatograpy.

Preferably, the chromatography used to separate any ACE2 present in the sample from ACE2 inhibiting agent(s), is selected from ion exchange chromatography and immunochromatography (eg using a specific anti-ACE2 binding agent such as anti-ACE2 polyclonal or monoclonal antibodies or fragments thereof such as Fab and F(ab″)₂ fragments, or recombinant antibody fragments such as scFv).

More preferably, the chromatography used to separate any ACE2 present in the sample from ACE2 inhibiting agent(s) is ion exchange chromatography.

Using ion exchange chromatography, any ACE2 present in the sample is bound to the ion exchange column, while the remainder of the sample (ie including ACE2 inhibiting agent(s)) passes through the column. The bound ACE2 is then eluted from the column, collected and assayed. In one particular embodiment, wherein a 5 ml HighTrap ANX Fast-Flow ion exchange column (Amersham Biosciences, Buckinghamshire, UK) is used, it has been found that the first 2.5 ml of sample to be eluted from the column with 5 ml of a high salt buffer (ie including 1 M NaCl) comprises the majority of ACE2 activity.

It is also to be understood that the method of the invention also extends to the use of the ANX Fast-Flow resin to extract ACE2 in a method whereby the proteins present in the sample are permitted to bind to the resin by gentle rocking, the resin then precipitated by centrifugation, the supernatant then discarded and the proteins bound to the resin subsequently eluted by re-suspending the resin in a high salt buffer (ie including 1 M NaCl). The resin is then again precipitated by centrifugation and the supernatant harvested and assayed for ACE2 catalytic activity.

Preferably, the step of detecting the level of ACE2 in the treated sample involves detecting ACE2 activity by, for example, measurement of cleavage or consumption of a detectably-labelled substrate of ACE2, or otherwise involves quantitatively detecting the presence of ACE2 by, for example, the use of a specific anti-ACE2 binding agent (eg an ELISA using anti-ACE2 polyclonal or monoclonal antibodies or fragments thereof).

In the step of detecting the level of ACE2 in the treated sample, the detection of an elevated level of ACE2 indicative of aberrant cardiac function will preferably be the detection of at least 5 ng/ml of ACE2 and, more preferably, at least 10 ng/ml of ACE2.

The detected ACE2 may be full-length ACE2 or a truncated form thereof (eg a truncated form of ACE2 retaining catalytic activity or retaining epitope(s) recognised by anti-ACE2 polyclonal or monoclonal antibodies or fragments thereof).

The method of the invention may be used either on its own or in combination with other suitable assays well known to persons skilled in the art (eg assays for peak creatine kinase and troponin I levels in cardiac injury as described by Fox et al, (2004)), in the diagnosis or assessment of risk of aberrant cardiac function in the subject.

As used herein, the term “aberrant cardiac function” refers to any condition affecting the proper functioning of the heart leading to heart disease, in particular ischaemic heart disease which is characterised by a deficiency of blood flow to the heart (eg angina), or which is otherwise characterised by damage to the heart muscle (myocardium) such as myocardial infarction (MI).

Accordingly, the method of the invention is particularly suited to the diagnosis or assessment of risk of aberrant cardiac function in subjects showing traditional heart disease risk factors such as obesity, hypercholesterolaemia and/or a familial history of heart disease. The method of the invention is also suitable for monitoring the risk of further aberrant cardiac function in subjects who have undergone procedures such as surgical insertion of a stent or angioplasty. Thus, the method may be used to monitor the success of such surgical treatments.

In a second aspect of the present invention, there is provided a process of separating metallopeptidase angiotensin-converting enzyme 2 (ACE2) from ACE2 inhibiting agent(s) in a sample from a subject, said process comprising the use of chromatography to separate any ACE2 present in said sample from ACE2 inhibiting agent(s).

Preferably, the sample subjected to the process of the invention is a plasma sample.

Preferably, the chromatography used in the process of the invention to separate any ACE2 present in the sample from ACE2 inhibiting agent(s) is ion exchange chromatography.

It is also considered that the detection of elevated levels of ACE2 in a sample such as plasma from a subject, is generally indicative of hypoxia in the subject. Thus, it is to be understood that the present invention extends to methods of assessing diseases and conditions characterised by hypoxia (ie in addition to hypoxia caused through aberrant cardiac function) such as pulmonary fibrosis, pulmonary oedema, and diseases and conditions characterised by haemoglobin deficiency, wherein such methods comprise the steps of:
(i) providing a sample from a subject;
(ii) treating said sample to
   separate any metallopeptidase angiotensin-converting enzyme 2 (ACE2) present in said sample from ACE2 inhibiting agent(s), or otherwise to
   remove or inactivate ACE2 inhibiting agent(s); and
(iii) detecting the level of ACE2 in said treated sample.

In a third aspect, the present invention provides a kit for detecting the level of metallopeptidase angiotensin-converting enzyme 2 (ACE2) in a sample from a subject, said kit comprising:
(i) means for separating any ACE2 present in said sample from any ACE2 inhibiting agent(s) in said sample, or otherwise for removing or inactivating any ACE2 inhibiting agent(s) in said sample; and
(ii) means for detecting the level of ACE2 in said sample.

The sample mentioned in this third aspect, may be as described above.

Preferably, the means for separating any ACE2 from ACE2 inhibiting agents(s) provided in the kit is a chromatography column, more preferably, an ion exchange chromatography column. Typically, such chromatography columns will be provided in a form readily amenable for inclusion in a kit (eg miniaturised forms). Such forms will be well known to persons skilled in the art.

In another embodiment of the kit of the invention, the means for separating any ACE2 from ACE2 inhibiting agent(s) may comprise a chromatography resin provided in a tube or container capable of being centrifuged. For example, the chromatography resin may be present in a 10 ml centrifugation tube to which sample is then added after which the proteins present in the sample (such as ACE2) are permitted to bind to the resin by gentle rocking before precipitation of the resin by centrifugation. The supernatant (including ACE2 inhibiting agent(s)) can be discarded and the bound proteins eluted.

Accordingly, it is preferred that the kit further comprise a high salt buffer (ie including 1M NaCl) which facilitates elution of bound ACE2 from the column/chromatography resin.

In a further embodiment of the kit of the invention, the means for separating any ACE2 from ACE2 inhibiting agent(s) may comprise an agent such as a specific anti-ACE2 binding agent (eg an anti-ACE2 polyclonal or monoclonal antibody or fragment thereof as described above). Such agents are appropriate for use in an ELISA based assay format.

In yet a further embodiment of the kit of the invention, the means for separating any ACE2 from ACE2 inhibiting agent(s) may be in the form of a dip-stick provided with a specific anti-ACE2 binding agent.

The means for detecting the level of ACE2 provided in the kit may comprise, for example, a detectably-labelled substrate which permits measurement of ACE2 activity by enabling the measurement of its cleavage or consumption. The substrate may be labelled so as to produce a fluorescent or coloured product when cleaved. This may be detected by eye which can then be compared with a colour chart to give an approximate indication of the level of ACE2 present in the sample. Other suitable detection methods which are amenable to being provided in a kit will be well known to persons skilled in the art.

In a fourth aspect, the present invention provides a method of assessing a subject for aberrant cardiac function by use of a kit according to the third aspect of the invention.

In order that the nature of the present invention may be more clearly understood, preferred forms thereof will now be described with reference to the following non-limiting example.

### EXAMPLE 1

Materials and Methods

Chromatographic Procedure for Separation of ACE2 from Inhibitor

Blood collected from patients is treated with lithium heparin as an anti-coagulant, and the red cells removed by centrifugation. Plasma is then stored at −70° C. until processed. Plasma is thawed when required and 0.25 ml is diluted in 5 ml buffer A (20 mM Tris-HCI, pH 6.5). The diluted sample is loaded onto a disposable 5 ml HighTrap ANX Fast-Flow ion exchange column (Amersham Biosciences, Buckinghamshire, UK), which is pre-equilibrated with buffer A. Using a syringe, steady pressure is applied to the column such that the flow rate is approximately 1 ml/min. Once the sample has passed through the column, the column is washed with 5 ml of buffer A. Bound protein (including ACE2) is eluted from the column with 5 ml buffer B (Buffer A+1 M NaCl). The first 2.5 ml to elute from the column is reserved as the majority of the ACE2 activity elutes in this fraction.

Suspension Procedure for Separation of ACE2 from Inhibitor

In an alternative procedure for separating ACE2 from ACE2 inhibiting agent(s) in plasma, plasma is collected and stored as described above; when thawed, 0.25 mL is diluted into 1.2 ml low ionic strength buffer (Buffer A), and added to 200 μl ANX Fast-Flow resin (Amersham Biosciences, Buckinghamshire, UK) which has previously been washed with the low ionic strength buffer. Plasma proteins are allowed to bind to the resin by gentle rocking for 30 min, then the resin is precipitated by a low speed centrifugation. The supernatant is discarded, and the resin washed in 1.2 ml fresh buffer for 10 min before a second centrifugation. This supernatant is also discarded, and proteins are eluted with 1.2 ml high salt buffer (ie. Buffer A+1 M NaCl ) (10 min rocking, followed by centrifugation).

Quenched Fluorescent Substrate Assay of ACE2 Activity

ACE2 catalytic activity was measured by cleavage of a quenched fluorescent substrate (QFS), namely (7-methoxycoumarin-4-yl)-acetyl-Ala-Pro-Lys(2,4-dinitrophenyl) as published in Vickers et al, 2002. The substrate is kept as stock at 5 mg/ml (7.14 mM) at −20° C. in DMSO, diluted 14.28-fold in ACE2 buffer (100 mM Tris-HCl, 1 M NaCl, pH 6.5) for a 500 μM working stock for assay. Assays were performed in black 96-well microtiter plates with 100 μl/well sample and ACE2 buffer in a final volume of 200 μl per well. In a separate series of wells, 100 μl/well sample, 60 μl/well ACE2 buffer, and 20 μl/well of the specific ACE2 activity inhibitor, MLN-4760 (Millenium Pharmaceuticals, Cambridge, Mass., USA) at 100 mM is added. 50 μM QFS (20 μl/well) is then added to all wells.

Reactions proceeded at 37° C. within a temperature-controlled fluorescence microplate reader (FLUOStar Optima, BMG Labtech, Offenburg, Germany) and continuous readings of the liberated fluorescence taken over a 4 hr period (λex=320 nm, λem =405 nm). Specific ACE2 activity was calculated by subtraction of fluorescence in the presence of MLN-4760 inhibitor from the fluorescence in the absence of the MLN-4760 inhibitor.

Shedding of ACE2 from Chinese Hamster Ovary Cells

Full-length human ACE2 was expressed in Chinese hamster ovary (CHO) cells using a modified DEAE-Dextran transfection method. Forty-eight hours post-transfection, cells were washed and placed in fresh medium ± phorbol ester (PMA, 1 μM). Medium was collected after 4 hr incubation and assayed for the presence of ACE2 activity.

Inhibition of ACE2 by Plasma

Medium collected from CHO cells expressing a soluble, secreted form of ACE2 (lacking the transmembrane and cytoplasmic domains) was used as a source of the enzyme. The inhibitory effect of human plasma (collected onto sodium citrate, ie no EDTA) on ACE2 activity was assessed by QFS assay.

Subjects

All subjects were in-patients and blood was taken within 24 h of the acute episode of chest pain. Control subjects were healthy volunteers with no known current or past history of cardiovascular disease.

### Results

ACE2 Activity is Inhibited by Plasma

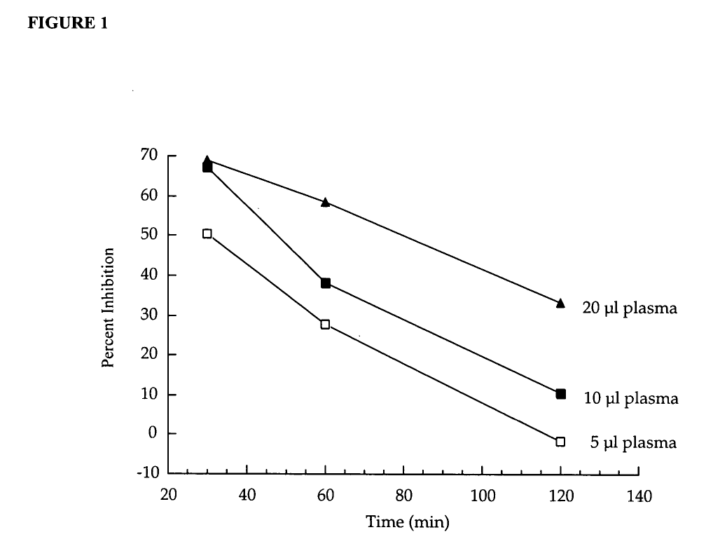

Catalytically active ACE2 has not been previously detected in human plasma, suggesting that circulating levels are extremely low or non-existent. The present applicants have, however, found that the addition of plasma to recombinant human ACE2 expressed in CHO cells (either the full-length, membrane-bound form or a soluble, secreted form) markedly inhibits the cleavage of an ACE2 quenched fluorescent substrate (FIG. 1) thereby suggesting that previous attempts to detect ACE2 in human plasma may have been due to the presence of an ACE2 inhibiting agent (ie a “masking agent”).

The level of inhibition increases with the volume of plasma added, and does not reflect chelation of the catalytic zinc atom as EDTA is not used as an anti-coagulant. Further, the plasma does not inhibit another related zinc metallopeptidase, endothelin-converting enzyme, while other metallopeptidases normally present in plasma, including ACE, are fully active and readily detected. Thus, the results indicate that plasma contains a relatively specific and competitive inhibiting agent of ACE2. Initial observations on the physicochemical characteristics of this ACE2 inhibiting agent (data not shown) indicate that:
(i) it is of low molecular weight,
(ii) it is hydrophilic and cationic,
(iii) it remains soluble in 40% organic solvent,
(iv) it retains activity with only some loss of potency following heat treatment (60° C., 20 min), and
(v) it inhibits both membrane-bound and soluble, secreted forms of ACE2.

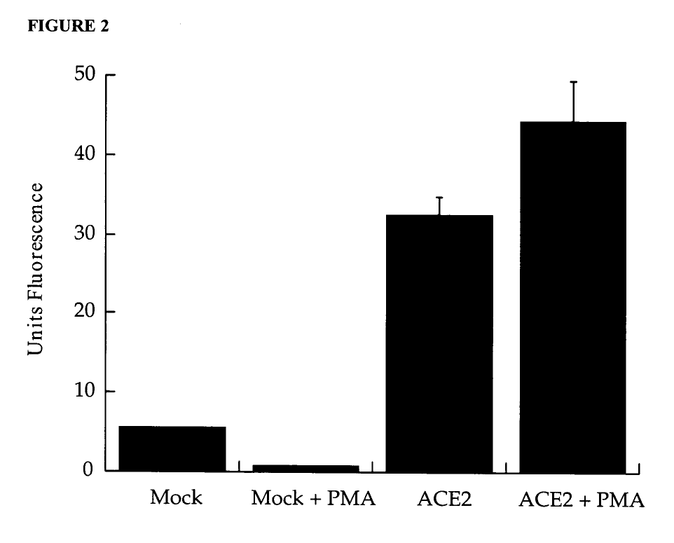

ACE2 release from the plasma membrane of CHO cells was enhanced by the addition of phorbol ester (PMA), which is known to stimulate the proteases involved in the secretion of plasma membrane proteins. By contrast, only low levels of QFS-cleaving activity was detected in medium from mock-transfected cells (FIG. 2).

ACE2 Activity is Elevated in Post-Myocardial Infarction Plasma

To verify that ACE2 is indeed present in plasma, a method was developed based on anion exchange chromatography to extract active ACE2 from plasma. As it is possible that the ACE2 QFS may be cleaved by other peptidases, the activity of ACE2 was also verified using the specific ACE2 activity inhibitor, MLN-4760, as well as by assay of ACE2 immunopurified from plasma using an anti-ACE2 antibody (R&D Systems, Minneapolis, Minn., USA). Alternative specific anti-ACE2 antibodies can be raised by immunising mice with a peptide derived from the extracellular domain (residues 107-116 of the human sequence) or cytoplasmic tail (residues 764-777 of the human sequence). The amino acid sequence of human ACE2 is described in Donoghue M et al, (2000) and Tipnis SR et al, (2000).

Plasma was collected from subjects following MI, as well as from age-matched healthy volunteers, and processed by anion exchange chromatography in order to remove the presence of the endogenous inhibiting agent of ACE2.

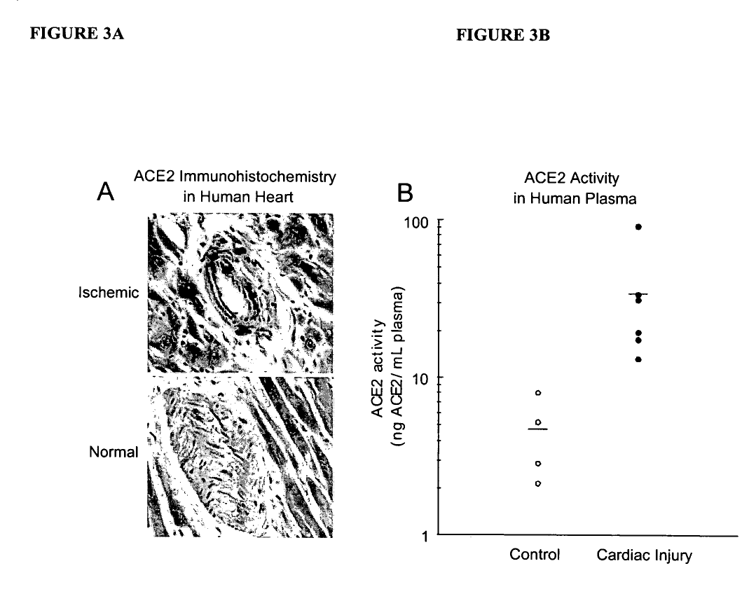

Preliminary results using this method show that while levels of plasma ACE2 are indeed low in normal subjects (4.72±0.47 ng solACE2/ml plasma, mean±s.e.m., n=5), the levels are markedly elevated following MI (40.1±17.6 ng/ml, mean±s.e.m., n=4) (FIG. 3A and B and Table 1).

All subjects were in-patients and blood was taken within 24 h of the acute episode of chest pain. ACE2 activity was as measured as the change in liberated fluorescence generated by cleavage of the ACE2 quenched fluorescent substrate (QFS) over a 3.8 hr time course at an excitation wavelength of 320 nm in the presence and absence of MLN-4760 inhibitor. The results presented below in Table 1 represent the average fluorescence measured in duplicate wells.

TABLE 1

| | |
|---|---|
| Subject No. | ng ACE2/ml plasma |

Normal

| | |
|---|---|
| 1 | 8.1 |
| 2 | 2.14 |
| 3 | 2.86 |
| 4 | 5.24 |
| 5 | 5.24 |

Post-myocardial infarction

| | |
|---|---|
| 1 | 31.4 |
| 2 | 19.5 |
| 3 | 17.6 |
| 4 | 91.9 |

Single-factor ANOVA comparing myocardial infarction vs. control results inp=0.06.

Plasma ACE2 is Elevated Following Myocardial Infarction

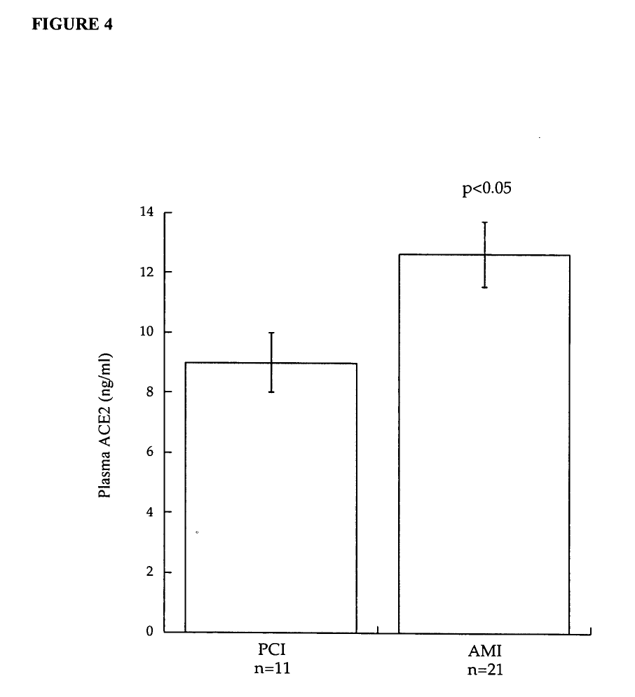

Using the described above, plasma ACE2 was measured in a total of 32 subjects. Subjects with documented acute myocardial infarction (AMI; n=21), as determined by conventional ECG analysis and blood troponin levels at least 50-fold above the normal range (<0.06 μg/L), had significantly higher plasma ACE2 levels than the control group consisting of patients entering the clinic to undergo percutaneous coronary intervention (PCI, angioplasty) in the absence of chest pain, abnormal ECG, and normal blood troponin levels (FIG. 4). Data is expressed as ng ACE2 per mL plasma, as derived from a standard curve generated by adding known amounts of purified recombinant ACE2 to heat-inactivated plasma prior to extraction and assay.

### Discussion

Both ACE and ACE2 are glycosylated, type I integral membrane proteins, each with a large extracellular catalytic domain, a single transmembrane region which anchors the enzyme to the plasmalemma, and a short cytoplasmic tail. It is now well-established that ACE is susceptible to proteolytic cleavage within the juxtamembrane region by one or more metalloproteases (“secretases”), resulting in the release of a soluble form present and active in plasma.

The present applicant has shown herein that ACE2 can also be proteolytically “shed” from the plasma membrane of both transiently transfected CHO cells and HepG2 cells (data not shown), a hepatic cell line which endogenously expresses the enzyme. In both cell types, shedding of ACE2 is increased up to two-fold by phorbol ester treatment (FIG. 2), as has been shown for other proteolytically secreted proteins including ACE, indicating that secretion of ACE2 can be regulated. The presence of ACE2 in plasma implies that this process occurs in vivo, although the cellular source(s) of the circulating enzyme is not yet known. However, as mentioned above, ACE2 expression is limited in the healthy individual to endothelial cells of the coronary and renal vasculatures, the renal tubules and the testis, and of these, the endothelial cells of the coronary vasculatures are the most likely source of MI-induced plasma ACE2.

The observation that ACE2 expression is markedly upregulated following MI, taken together with the data indicating the subsequent release of ACE2 into plasma, indicates a role for ACE2 in the cardiac response to an ischaemic event. Accordingly, detection of plasma ACE2 levels can be used as a biomarker of aberrant cardiac function or, more particularly, as an indicator of, for example, the type and extent of cardiac injury.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Donoghue M, Hsieh F, Baronas E, Godbout K, Gosselin M, Stagliano N, Donovan M, Woolf B, Robison K, Jeyaseelan R, Breitbart R E, Acton S A. A novel angiotensin-converting enzyme-related carboxypeptidase (ACE2) converts angiotensin I to angiotensin 1-9. Circ Res. 2000;87:E1-E9.
2. Fox K A, Birkhead J, Wilcox R, Knight C, Barth J. British Cardiac Society Working Group on the definition of myocardial infarction. Heart 2004; 90:603-9.
3. Ramchandran R, Kasturi S, Douglas J G, Sen I. Metalloprotease-mediated cleavage secretion of pulmonary ACE by vascular endothelial and kidney epithelial cells. Am J Physiol. 1996; 271:H744-51.
4. Tipnis S R, Hooper N M, Hyde R, Karran E, Christie G, Turner A J. A human homolog of angiotensin-converting enzyme—Cloning and functional expression as a captopril-insensitive carboxypeptidase. J Biol Chem. 2000;275:33238-33243.
5. Vickers C, Hales P, Kaushik V, Dick L, Gavin J, Tang J, Godbout K, Parsons T, Baronas E, Hsieh F, Acton S, Patane M, Nichols A, Tummino P. Hydrolysis of biological peptides by human angiotensin-converting enzyme-related carboxypeptidase. J Biol Chem. 2002; 277:14838-43.

1. A method of assessing a subject for aberrant cardiac function, said method comprising the steps of:
(i) providing a sample from said subject;
(ii) treating said sample to
separate any metallopeptidase angiotensin-converting enzyme 2 (ACE2) present in said sample from ACE2 inhibiting agent(s), or otherwise to
remove or inactivate ACE2 inhibiting agent(s); and
(iii) detecting the level of ACE2 in said treated sample,
wherein detection of an elevated level of ACE2 in said treated sample, relative to typical levels of ACE2 found in identical samples from healthy subjects, is indicative of aberrant cardiac function.

2. A method according to claim 1, wherein the sample is selected from the group consisting of whole blood, plasma, serum, lymph, urine, cerebrospinal fluid and semen.

3. A method according to claim 1, wherein the sample is plasma.

4. A method according to claim 1, wherein the step of treating the sample to either separate any ACE2 present in the sample from ACE2 inhibiting agent(s), or otherwise to remove or inactivate ACE2 inhibiting agent(s), comprises chromatography.

5. A method according to claim 4, wherein the chromatography is ion exchange chromatography.

6. A method according to claim 1, wherein the ACE2 inhibiting agent(s) inhibits or prevents detection of ACE2 activity by measurement of cleavage or consumption of a detectably-labelled substrate of ACE2.

7. A method according to claim 1, wherein the step of detecting the level of ACE2 in the treated sample comprises detecting ACE2 by measurement of cleavage or consumption of a detectably-labelled substrate of ACE2.

8. A method according to claim 1, wherein the step of detecting the level of ACE2 in the treated sample comprises quantitatively detecting the presence of ACE2 by the use of a specific anti-ACE2 binding agent.

9. A process of separating metallopeptidase angiotensin-converting enzyme (ACE2) from ACE2 inhibiting agent(s) in a sample from a subject, said process comprising chromatography to separate any ACE2 present in said sample from ACE2 inhibiting agent(s).

10. A process according to claim 9, wherein the sample is selected from the group consisting of whole blood, plasma, serum, lymph, urine, cerebrospinal fluid and semen.

11. A process according to claim 9, wherein the sample is plasma.

12. A process according to claim 9, wherein the chromatography is ion exchange chromatography.

13. A method of assessing a disease or condition characterised by hypoxia in a subject, wherein the method comprises the steps of:
(i) providing a sample from a subject;
(ii) treating said sample to
separate any metallopeptidase angiotensin-converting enzyme 2 (ACE2) present in said sample from ACE2 inhibiting agent(s), or otherwise to
remove or inactivate ACE2 inhibiting agent(s); and
(iii) detecting the level of ACE2 in said treated sample.

14. A method according to claim 13, wherein the disease or condition is selected from the group consisting of pulmonary fibrosis, pulmonary oedema, and diseases and conditions characterised by haemoglobin deficiency.

15. A kit for detecting the level of metallopeptidase angiotensin-converting enzyme 2 (ACE2) in a sample from a subject, said kit comprising:
(i) means for separating any ACE2 present in said sample from any ACE2 inhibiting agent(s) in said sample, or otherwise for removing or inactivating any ACE2 inhibiting agent(s) in said sample; and
(ii) means for detecting the level of ACE2 in said sample.

16. A kit according to claim 15, wherein the means for separating ACE2 from ACE2 inhibiting agent(s) is a chromatography column or chromatography resin.

17. A kit according to claim 15, wherein the means for separating ACE2 from ACE2 inhibiting agent(s) is a specific anti-ACE2 binding agent.

18. A kit according to claim 15, wherein the means used for separating ACE2 from ACE2 inhibiting agent(s) is a dip-stick provided with a specific anti-ACE2 binding agent.

19. A kit according to claim 15, wherein the means for detecting the level of ACE2 is a detectably-labelled substrate which permits measurement of ACE2 activity by enabling the measurement of its cleavage or consumption.

20. A method of assessing a subject for aberrant cardiac function by use of a kit according to claim 15.
